(19) United States
(12) Patent Application Publication
(10) Pub. No.: US 2022/0039055 A1
(43) Pub. Date: Feb. 3, 2022

(54) Aggregation of Beacon Data for Network Load Mitigation

(71) Applicant: ZEBRA TECHNOLOGIES CORPORATION, Lincolnshire, IL (US)

(72) Inventors: Douglas C. Bowman, Capitola, CA (US); Richard Lawerance Woodburn, Cupertino, CA (US); Edward W. Geiger, San Martin, CA (US); Yu Wan, Cupertino, CA (US); Janakiraman Gopalan, Cupertino, CA (US); Thomas E. Warner, Moraga, CA (US); Eric T. Tokubo, Newark, CA (US); Carl S. Mower, San Jose, CA (US)

(21) Appl. No.: 16/944,723

(22) Filed: Jul. 31, 2020

(51) Int. Cl. H04W 64/00; H04W 76/11; H04W 76/15; G16H 50/80; H04W 4/029

(52) U.S. Cl. CPC H04W 64/006; H04W 64/003; H04W 4/029; H04W 76/15; G16H 50/80; H04W 76/11

(57) ABSTRACT

A method in a computing device includes: via a short-range interface of the computing device, detecting a plurality of beacons emitted by another computing device, each beacon containing an identifier of the other computing device; obtaining respective proximity indicators corresponding to the beacons; generating a first set of aggregated attributes from the proximity indicators; storing the first set of aggregated attributes in association with the identifier of the other computing device; and transmitting the first set of aggregated attributes to a server configured to detect physical proximity between the computing device and the other computing device based on the first set of aggregated attributes.

### BACKGROUND

Contact tracing systems may enable individual mobile device operators to be notified of potential exposure to conditions such as infections diseases. In such systems, the mobile devices may both emit beacons, and listen for beacons from other mobile devices. Physical proximity between the devices (and therefore the operators of those devices) may be determined, e.g. at a server, from various properties of the beacons. In some implementations, however, reporting of beacon detections to the server may negatively affect network performance.

### BRIEF DESCRIPTION OF THE SEVERAL VIEWS OF THE DRAWINGS

The accompanying figures, where like reference numerals refer to identical or functionally similar elements throughout the separate views, together with the detailed description below, are incorporated in and form part of the specification, and serve to further illustrate embodiments of concepts that include the claimed invention, and explain various principles and advantages of those embodiments.

FIG. 1 is a schematic of a system for detecting potential exposure to conditions such as infectious diseases.

FIG. 2 is a block diagram of certain internal components of a mobile computing device of the system of FIG. 1.

FIG. 3 is a flowchart of a method of aggregating beacon data for reporting.

FIG. 4A is a diagram illustrating an example performance of blocks 310 and 320 of the method of FIG. 3.

FIG. 4B is a diagram illustrating an example performance of blocks 310 and 330 of the method of FIG. 3.

FIG. 5 is a diagram illustrating the output of further performances of blocks 310 and 330 of the method of FIG. 3.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Examples disclosed herein are directed to a method in a computing device, the method including: via a short-range interface of a computing device, detecting a plurality of beacons emitted by another computing device, each beacon containing an identifier of the other computing device; obtaining respective proximity indicators corresponding to the beacons; generating a first set of aggregated attributes from the proximity indicators; storing the first set of aggregated attributes in association with the identifier of the other computing device; and transmitting the first set of aggregated attributes to a server configured to detect physical proximity between the computing device and the other computing device based on the first set of aggregated attributes.

Additional examples disclosed herein are directed to a computing device, comprising: a first short-range communications interface; a memory; and a controller configured to: via the short-range interface, detect a plurality of beacons emitted by another computing device, each beacon containing an identifier of the other computing device; obtain respective proximity indicators corresponding to the beacons; generate a first set of aggregated attributes from the proximity indicators; store the first set of aggregated attributes in association with the identifier of the other computing device; and transmit the first set of aggregated attributes to a server configured to detect physical proximity between the computing device and the other computing device based on the first set of aggregated attributes.

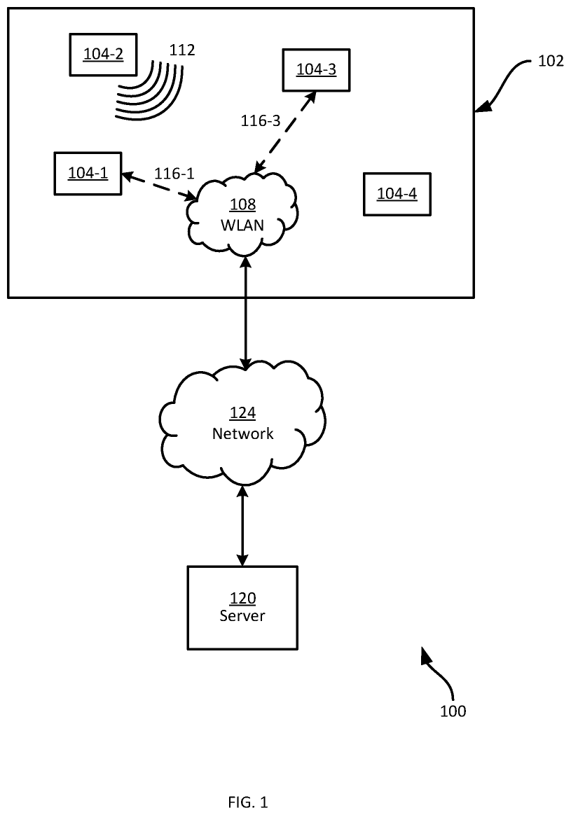

FIG. 1 illustrates a system 100 for detecting potential exposure to conditions such as infectious diseases, for example within a facility 102 such as a healthcare facility (e.g. a hospital), a transport and logistics facility (e.g. a warehouse), or the like. The system 100 may therefore also be referred to as a contact tracing system 100.

The system 100 includes a plurality of mobile computing devices 104, of which four examples 104-1, 104-2, 104-3, and 104-4 are shown in FIG. 1. As will be apparent to those skilled in the art, the system 100 may include smaller or greater numbers of mobile devices 104, e.g. based on the number of workers within the facility 102. Each device 104 is a computing device such as a smart phone, a tablet computer, or the like. The devices 104 may also, however, be implemented as devices with limited computational resources in comparison with those of smartphones, tablet computers and the like. In the present example, the devices 104 are bridge devices enabled to communicate over two interfaces, such as a short-range interface (e.g. Bluetooth™), and a second interface with a wireless network such as the wireless local area network (WLAN) 108 shown in FIG. 1. Such bridge devices may be low-cost and therefore have limited storage and data processing capabilities in comparison with smart phones and the like.

Each device 104 may be carried by an operator (e.g. a worker in the facility 102), e.g. alone or along with other computing devices worn or carried by the operator. The device 104 periodically emits a beacon using the short-range interface mentioned above. The interval between beacons is configurable. For example, each device 104 can be configured to emit a beacon at one-second intervals. Different devices 104 may use different beacon intervals, however. Each device 104 also scans for beacons emitted by the other devices 104. Thus, for example, the device 104-2 emits a beacon 112, which may be detected by the devices 104-1 and 104-3. The device 104-4 may be too distant from the device 104-2 to detect the beacon 112. The devices 104-1 and 104-3, responsive to detecting the beacon 112, can be configured to report, via wireless connections 116-1 and 116-3 to the WLAN 108, attributes obtained from the beacon 112.

In particular, the devices 104-1 and 104-3 may report the above-mentioned attributes to a server 120 via the WLAN 108 and, in some examples, a network 124 (e.g. a wide area network, which may include the Internet). In other examples, the server 120 may be located at the facility 102, and connected directly to the WLAN 108. In any event, the server 120 can be configured to process the data reported by the devices 104 and determine whether any of the devices 104 have been within a predefined proximity of one another (e.g. within about two meters). For instance, the devices 104-1 and 104-3 may report an indication of signal strength associated with the beacon 112 to the server 120, and the server 120 can estimate a distance between the device 104-2 and each of the devices 104-1 and 104-3 when the beacon 112 was detected by those devices 104.

The server 120 can also, when a pair of devices 104 are determined to have come within the predefined proximity mentioned above, transmit a message to the relevant devices 104 themselves, or other computing devices associated with the operators of the affected devices 104. In some examples, transmission of the above messages may be performed only when the server 120 receives (e.g. from a separate source, not illustrated in FIG. 1) an indication that the operator of one of the affected devices 104 has been diagnosed with an infectious disease, such that the other operators may have been exposed to the infectious disease by proximity.

The above-mentioned proximity detection and notification functionality need not be performed by the server 120 itself. For example, the server 120 can host a data lake or other form of repository, while a further computing device retrieves data from the repository and performs the contact tracing functionality.

As will be apparent to those skilled in the art, each device 104 emits beacons as well as detecting beacons emitted by other devices 104. The total number of beacons detected across the set of devices 104 deployed in the facility 102 may therefore be sufficient to cause congestion or outages of the WLAN 108. The devices 104 are therefore, as discussed in detail herein, configured to generate and report aggregated attributes derived from detected beacons, rather than the individual beacon detections. The aggregated reporting processes implemented by the devices 104 may enable the server 120 to continue performing the contact tracing functionality mentioned above, while reducing the operational load on the WLAN 108. Further, the aggregated reporting processes may be deployed with relatively low-cost devices such as the bridge devices mentioned above.

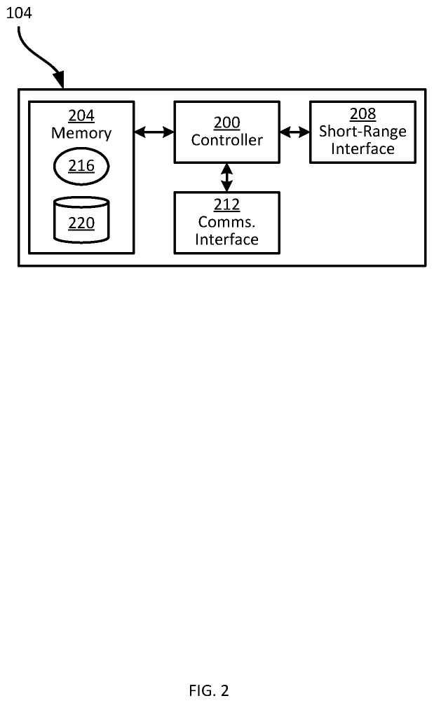

Turning to FIG. 2, certain internal components of a device 104 are illustrated. Each of the devices 104 in the system 100 include the components shown in FIG. 2, although the devices 104 need not have a uniform form factor, computational capabilities, or the like.

The device 104 includes a controller 200, such as a central processing unit (CPU), application-specific integrated circuit (ASIC) or the like, coupled with a non-transitory computer readable storage medium, such as a memory 204. The memory 204 can include a suitable combination of volatile memory (e.g. Random Access Memory or RAM) and non-volatile memory (e.g. read only memory or ROM, Electrically Erasable Programmable Read Only Memory or EEPROM, flash memory). The controller 200 and the memory 204 each comprise one or more integrated circuits.

The device 104 also includes a short-range communications interface 208, such as a Bluetooth™ interface, enabling the device 104 to communicate with other devices, and also to emit and detect beacons as summarized in connection with FIG. 1. The short-range interface 208 may implement the Bluetooth Low Energy (BLE) protocol(s), for example. The device 104 also includes an additional communications interface 212, such as an interface enabling wireless communication with other computing devices via WiFi or the like.

The memory 204 can store computer readable instructions for execution by the controller 200. In the present example, the memory 204 stores a data aggregation application 216 which, when executed by the controller 200, configures the controller 200 to detect beacons from other devices 104 and generate aggregated attributes corresponding to those beacons for reporting to the server 120. The memory 204 also stores a repository, which may also be referred to as a buffer 220, in which aggregated attributes are stored for subsequent reporting to the server 120.

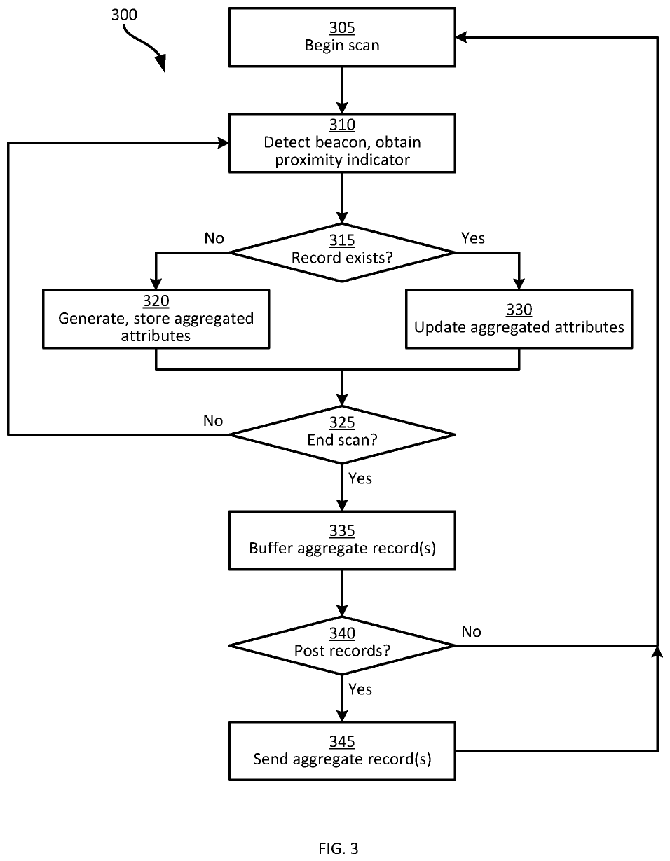

Turning to FIG. 3, a method 300 of aggregating beacon data for reporting is illustrated. The method 300 will be described below in conjunction with its performance in the system 100, and in particular by a device 104. The example performance of the method 300 discussed below is performed by the device 104-1 shown in FIG. 1. As will be apparent, however, each device 104 in the system 100 may be configured to perform the method 300, via execution of the application 216.

At block 305, the device 104-1 is configured to begin scanning for beacons from other devices 104. For example, the short-range interface 208 can be configured to initiate a scan window, in which the interface 208 is employed to listen for beacons from other devices 104 for a predetermined time period (e.g. 30 seconds).

At block 310, the device 104-1 is configured to detect a beacon emitted by another device 104. In the present example performance of block 310, it is assumed that the device 104-1 detects, via the short-range interface 208, the beacon 112 emitted by the device 104-2, as shown in FIG. 1. The beacon 112 contains, for example, an identifier of the emitting device 104-1. In some examples, a plurality of identifiers can be included, such as the unique identifier (UUID), Major, and Minor identifying values specified under the BLE protocol. The beacon 112 may also contain a timestamp in some examples.

At block 310, the device 104-1 is also configured to obtain a proximity indicator corresponding to the beacon 112. The proximity indicator is an indication of the distance between the device 104-1 itself and the device 104-2 that emitted the beacon 112. The proximity indicator can be, for example, an indication of signal strength associated with the beacon 112. For example, the device 104-1 can determine a received signal strength indicator (RSSI) from the beacon 112, such as a value between zero and one hundred, a value between −100 and zero, or the like, with greater values indicating greater received signal strength.

Having detected the beacon 112 and determined the proximity indicator, the device 104-1 can store a record, e.g. in the memory 204, containing the proximity indicator and the identifier(s) of the device 104-2 extracted from the beacon 112.

At block 315, the device 104-1 is configured to determine whether a record to track beacons from the device 104-2 (i.e. from the emitter of the beacon detected at block 310) exists in the memory 204. The determination at block 315 is affirmative if a previous performance of block 310 within the same scan window detected a beacon from the same device (104-2, in this example). If the beacon detected at block 310 is the first beacon from the device 104-2 in the current scan window, the determination at block 315 is negative. In the present example, it is assumed that the beacon 112 is the first beacon from the device 104-2 detected in the scan window initiated at block 305.

Following a negative determination at block 315, therefore, the device 104-1 proceeds to block 320. At block 320, the device 104-1 generates a set of aggregated attributes from the beacon detected at block 310. As will be discussed in greater detail below, the attributes generated at block 320 are also updated based on subsequent beacons detected from the device 104-2, such that the set of attributes can represent a plurality of individual beacons from a given emitting device.

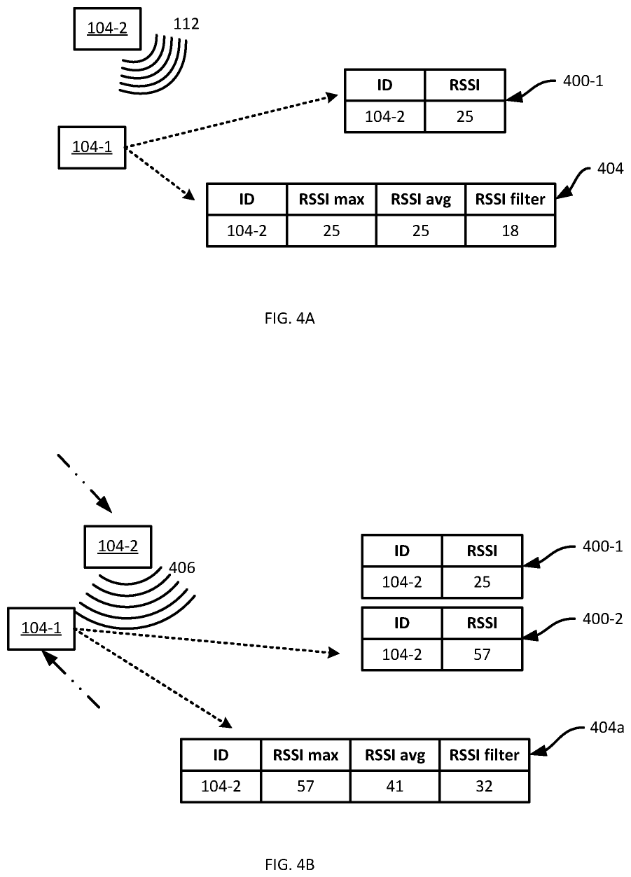

The aggregated attributes generated at block 320 can include a variety of attributes based on the proximity indicator (e.g. RSSI) obtained at block 310. For example, turning to FIG. 4A, the devices 104-1 and 104-2 are shown as arranged in FIG. 1, with the beacon 112 being emitted by the device 104-2 and detected by the device 104-1. The device 104-1, responsive to detecting the beacon 112, may store a beacon record 400-1 in the memory 204, as noted earlier, containing the identifier of the device 104-2, and the proximity indicator determined from the beacon 112.

FIG. 4A also illustrates an aggregated beacon record 404 created at block 320. The aggregated record 404 contains an identifier of the beacon emitter (the device 104-2, in this example), and the above-mentioned aggregated attributes. In the illustrated example, the aggregated attributes include a maximum proximity indicator, an average proximity indicator, and a filter value. Each of the above attributes are derived from the set of beacons detected from the same emitting device throughout the current scan window. The filter value can be the output of a linear smoothing filter process implemented by the controller 200. For example, the filter process can include adding a fraction of the current proximity indicator to a fraction of the previous filter output (representing any previous proximity indicators). In some examples, the filter may be a linear approximation of an infinite impulse response (IIR) filter. The filter process may also be configured to bias the output in favor of increases in the proximity indicator over decreases in the proximity indicator. That is, when the current proximity indicator is greater than a previous proximity indicator (or than the previous filter output), the fraction of the current proximity indicator added to the filter output may be greater than the fraction employed if the current proximity indicator is lower than the previous proximity indicator or filter output.

Various other aggregated attributes can be employed in addition to, or instead of, those shown in FIG. 4A. For example, the aggregated attributes can include the second-highest proximity indicator from the corresponding emitting device. In further examples, the aggregated attributes can include a count of beacons detected from the emitting device within the current scan window.

Returning to FIG. 3, having generated the aggregated set of attributes and stored them in the record 404, the device 104-1 proceeds to block 325. At block 325, the device 104-1 determines whether the scanning initiated at block 305 is to be terminated. For example, the device 104-1 can determine whether the predefined scan window (e.g. of 30 seconds, as noted earlier) has expired. In the present example performance of the method 300, it is assumed that the determination at block 325 is negative. The device 104-1 therefore returns to block 310 to await detection of a further beacon.

Referring to FIG. 4B, the devices 104-1 and 104-2 are again shown in isolation, having moved relative to one another as indicated by the dot-dash arrows. Such movement can result from the operators of the devices 104-1 and 104-2 travelling through the facility 102. The device 104-2 is shown emitting a further beacon 406, which is detected by the device 104-1 at a further performance of block 305. Detection of the beacon 406 by the device 104-1 also leads to determination of a further proximity indicator, such as the RSSI value of 57, and the storage of the proximity indicator in a beacon record 400-2. At block 315, the determination is affirmative, because the record 404 (containing an identifier matching the identifier in the beacon record 400-2) was created via the performance of block 315 shown in FIG. 4A.

Therefore, the device 104-1 proceeds to block 330. At block 330, the device 104-1 updates the aggregated attributes in the record 404. As shown in FIG. 4B, the record 404 is updated to a record 404a, which contains the identifier of the device 104-2, as well as an updated set of aggregated attributes. In particular, the maximum proximity indicator has been replaced with the proximity indicator derived from the beacon 406, the average proximity indicator is the average of the two proximity indicators of the records 400-1 and 400-2, and the filter value is updated based on the newly detected beacon 406.

As will now be apparent, the device 104-1 may perform numerous further instances of blocks 310, 315 and 320 or 330 during a given scan window, accumulating data for a plurality of beacons from various other devices 104. The device 104-1 generates an aggregated record for each other device 104 and updates the attributes therein with each subsequently detected beacon from that device 104.

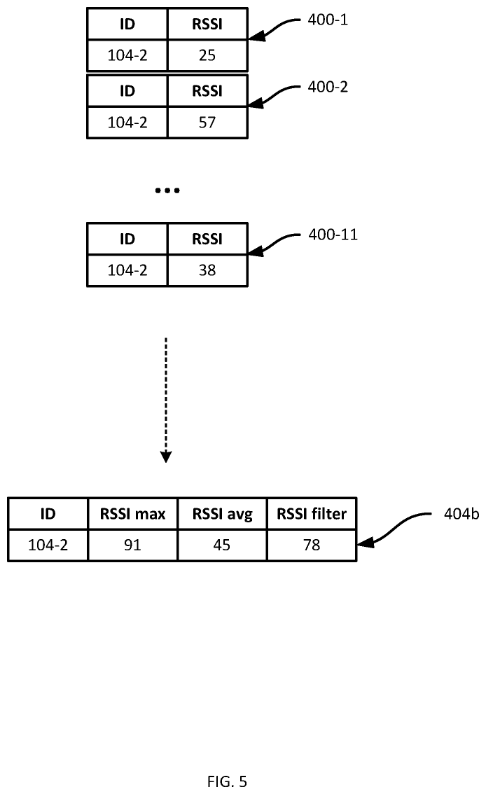

Turning to FIG. 5, the result of a series of additional performances of blocks 310, 315 and 330 at the device 104-1 for beacons detected from the device 104-2 is illustrated. In particular, a total of eleven beacons are assumed to have been detected from the device 104-2 in the current scan window, leading to additional beacon records 400 up to and including the record 400-11. The device 104-1 therefore generates a further updated aggregated record 404b containing the above-mentioned aggregated attributes, which represent the full set of beacons detected from the device 104-2 during the scan window. As will now be apparent, the aggregated record 404b represents the set of individual beacon records 400 with lower storage requirements than the records 400 themselves.

Returning to FIG. 3, when the scan window has ended (i.e. the determination at block 325 is affirmative, the device 104-1 proceeds to block 335. At block 335, the device 104-1 is configured to store the aggregated record 404b (as well as any aggregated records representing beacons from other devices 104) in the buffer 220. The device 104-1 can also discard any individual beacon records 400 that were stored in the memory 204. In some examples, the aggregated record 404 may be generated only once, at the end of the scan window. That is, the aggregated attributes can be generated at block 335 for each device 104 from which beacons were detected, rather than being updated continuously throughout the scan window.

Following storage of the aggregated record(s), the device 104-1 proceeds to block 340. At block 340, the device 104-1 is configured to determine whether to transmit (which may also be referred to as reporting, or posting), the aggregated records to the server 120. The determination at block 340 can be based on a predefined post interval, e.g. according to which aggregated records are posted every five minutes (although both shorter and longer time periods may also be employed). In other examples, the determination at block 340 can be based on network conditions within the WLAN 108, such as signal strength, congestion levels, and the like. In further examples, the determination at block 340 can be based on storage capabilities of the device 104-1. For example, the determination at block 340 can be affirmative when the available storage capacity in the buffer 220 falls below a threshold relative to the total capacity (e.g. 10%).

When the determination at block 340 is negative, the device 104-1 returns to block 305 to initiate the next scan window. The next scan window can immediately follow the termination of the current scan window, but in some implementations scan windows can be separated by a configurable time period.

When the determination at block 340 is affirmative, the device 104-1 proceeds to block 345. At block 345, the device 104-1 transmits the aggregated record(s) in the buffer 220 to the server 120, via the network 108 (and the network 124, as needed). The device 104-1 may then discard the aggregated record(s), releasing the buffer 220 for the next performance of the method 300.

As will now be apparent, the transmission of the aggregated records, instead of individual beacon records, can reduce the volume of data transmitted by each device 104 to the server 120. In particular, the size of each aggregated record is substantially constant, regardless of the number of individual beacons detected from a given device 104. The aggregated attributes, however, still represent the full set of beacons detected from that device. The representation of beacons from a device 104 by the aggregated attributes is likely to be imperfect, but the loss in fidelity of representation is balanced by a potentially significant reduction in resource usage at the devices 104 and in network traffic within the WLAN 108.

Further variations to the above are contemplated. For example, each device 104 may evaluate each beacon detected at block 310 before proceeding to block 315. For example, the device 104 may determine whether the proximity indicator is above a predefined threshold. When the proximity indicator does not exceed the threshold, the beacon may simply be discarded, and a further performance of block 310 may be initiated. That is, beacons detected with insufficient signal strength may be ignored, and therefore not represented in the aggregated attributes. The threshold may be selected to exclude beacons that are likely to be sufficiently distant from the detecting device 104 as to not represent potential contacts for the purpose of the contact tracing function performed at the server 120.

In other examples, beacons may be discarded as above when the device identifiers contained therein fail to meet certain criteria. For example, the facility 102 may contain various types of devices that broadcast beacons, some of which may not be directly relevant to the above-mentioned contact tracing functionality. The devices 104 may therefore be configured to only process beacons from other devices having predefined identifiers (e.g. identifiers within a predefined range).

In the foregoing specification, specific embodiments have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

Moreover in this document, relational terms such as first and second, top and bottom, and the like may be used solely to distinguish one entity or action from another entity or action without necessarily requiring or implying any actual such relationship or order between such entities or actions. The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially”, “essentially”, “approximately”, “about” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way, but may also be configured in ways that are not listed.

It will be appreciated that some embodiments may be comprised of one or more specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

1. A method in a computing device, comprising:
via a short-range interface of the computing device, detecting a plurality of beacons emitted by another computing device, each beacon containing an identifier of the other computing device, wherein the computing device and the another computing device are configured to be mobile during operation;
obtaining respective proximity indicators corresponding to the beacons;
generating a first set of aggregated attributes from the proximity indicators;
storing the first set of aggregated attributes in association with the identifier of the other computing device; and
transmitting the first set of aggregated attributes to a server configured to detect physical proximity between the computing device and the other computing device based on the first set of aggregated attributes.

2. The method of claim 1, wherein storing the first set of aggregated attributes includes:
for each beacon, determining whether an aggregated record exists;
when the aggregated record does not exist, creating the aggregated record; and
when the aggregated record does exist, updating the aggregated record.

3. The method of claim 2, wherein updating the aggregated record includes replacing previously generated aggregated attributes with the first set of aggregated attributes.

4. The method of claim 1, further comprising:
prior to generating the first set of aggregated attributes, determining whether each proximity indicator exceeds a threshold;
when one of the proximity indicators does not exceed the threshold, discarding the proximity indicator.

5. The method of claim 1, further comprising:
responsive to storing the first set of aggregated attributes, discarding the proximity indicators.

6. The method of claim 1, wherein transmitting the first set of aggregated attributes includes controlling a second communications interface of the computing device to transmit the first set of aggregated attributes over a wireless network.

7. The method of claim 1, further comprising:
detecting the plurality of beacons during a first scan window;
storing the first set of aggregated attributes responsive to termination of the first scan window;
during a second scan window, generating and storing a second set of aggregated attributes based on a further plurality of beacons; and
transmitting the first set of aggregated attributes and the second set of aggregated attributes to the server.

8. The method of claim 7, further comprising: transmitting the first set of aggregated attributes and the second set of aggregated attributes to the server responsive to expiry of a post interval.

9. The method of claim 8, wherein the post interval has a duration greater than a scan window duration.

10. The method of claim 1, wherein the first set of aggregated attributes includes at least one of:
a maximum one of the proximity indicators,
an average of the proximity indicators, and
a filter output generated from the proximity indicators.

11. A computing device, comprising:
a short-range communications interface;
a memory; and
a controller configured to:
via the short-range interface, detect a plurality of beacons emitted by another computing device, each beacon containing an identifier of the other computing device wherein the computing device and the another computing device are configured to be mobile during operation;
obtain respective proximity indicators corresponding to the beacons;
generate a first set of aggregated attributes from the proximity indicators;
store the first set of aggregated attributes in association with the identifier of the other computing device; and
transmit the first set of aggregated attributes to a server configured to detect physical proximity between the computing device and the other computing device based on the first set of aggregated attributes.

12. The computing device of claim 11, wherein the controller is configured, in order to store the first set of aggregated attributes, to:
for each beacon, determine whether an aggregated record exists;
when the aggregated record does not exist, create the aggregated record; and
when the aggregated record does exist, update the aggregated record.

13. The computing device of claim 12, wherein the controller is configured, in order to update the aggregated record, to replace previously generated aggregated attributes with the first set of aggregated attributes.

14. The computing device of claim 11, wherein the controller is configured to:
prior to generation of the first set of aggregated attributes, determine whether each proximity indicator exceeds a threshold;
when one of the proximity indicators does not exceed the threshold, discard the proximity indicator.

15. The computing device of claim 11, wherein the controller is configured to:
responsive to storing the first set of aggregated attributes, discard the proximity indicators.

16. The computing device of claim 11, further comprising:
a second communications interface;
wherein the controller is configured, in order to transmit the first set of aggregated attributes, to control the second communications interface to transmit the first set of aggregated attributes over a wireless network.

17. The computing device of claim 11, wherein the controller is configured to:
detect the plurality of beacons during a first scan window;
store the first set of aggregated attributes responsive to termination of the first scan window;
during a second scan window, generate and store a second set of aggregated attributes based on a further plurality of beacons; and
transmit the first set of aggregated attributes and the second set of aggregated attributes to the server.

18. The computing device of claim 17, wherein the controller is configured to: transmit the first set of aggregated attributes and the second set of aggregated attributes to the server responsive to expiry of a post interval.

19. The computing device of claim 18, wherein the post interval has a duration greater than a scan window duration.

20. The computing device of claim 11, wherein the first set of aggregated attributes includes at least one of:
a maximum one of the proximity indicators,
an average of the proximity indicators, and
a filter output generated from the proximity indicators.
